(19) United States
(12) Patent Application Publication
(10) Pub. No.: US 2007/0155710 A1
(43) Pub. Date: Jul. 5, 2007

(54) Androgen Treatment in Females

(76) Inventors: Norbert GLEICHER, Chicago, IL (US); David H. Barad, Closter, NJ (US); Dwyn V. Harben, Bryn Mawr, PA (US)

Correspondence Address: BEEM PATENT LAW FIRM, 53 W. JACKSON BLVD., SUITE 1352, CHICAGO, IL, 60604-3787, US

(21) Appl. No.: 11/680,973

(22) Filed: Mar. 1, 2007

Related U.S. Application Data
(63) Continuation-in-part of application No. 10/973,192, filed on Oct. 26, 2004.
(63) Continuation-in-part of application No. 11/269,310, filed on Nov. 8, 2005.

(51) Int. Cl. A61K 31/57

(52) U.S. Cl. 514/177

(57) ABSTRACT

A method of improving cumulative embryo score may comprise administering an androgen to a human female, for example, DHEA, for at least about four consecutive months followed by harvesting and fertilizing oocytes and forming embryos. Between about 50 mg and about 100 mg of DHEA may be administered to a human female per day. Moreover, a method of increasing the quantity of fertilized oocytes in one cycle of in vitro fertilization may comprise administering an androgen to a human female for at least about four consecutive months, harvesting and fertilizing the oocytes. Furthermore, a method of increasing the quantity of day 3 embryos from one cycle of in vitro fertilization may comprise administering an androgen for at least about four consecutive months, harvesting and fertilizing the oocytes and forming day 3 embryos. A method of normalizing ovarian DHEA also may include administering an androgen for at least about four consecutive months. A method of increasing the rate and number of euploid oocytes may include administering an androgen for at least about four consecutive weeks. In addition, a method of increasing male fetus sex ratio may comprise raising baseline androgen levels in a female prior to or at time of embryo implantation.

This application is a continuation-in-part of application Ser. No. 10/973,192 (attorney docket number 0222-0002) filed on Oct. 26, 2004 and Ser. No. 11/269,310 (attorney docket number 0222-0003) filed on Nov. 8, 2005.

### BACKGROUND OF THE INVENTION

1. Field of the Invention

The present invention relates to a method of improving ovulation induction and embryo quality in women undergoing in vitro fertilization and other infertility treatments involving administering an androgen such as dehydroepiandrosterone prior to or during ovulation stimulation cycles. In addition, the invention relates to a method of increasing male fetus sex ratio.

2. Description of the Related Art

The application of assisted reproductive technology has revolutionized the treatment of all forms of infertility. The most common assisted reproductive technology is in vitro fertilization (IVF), in which a woman's eggs are harvested and fertilized with a man's sperm in a laboratory. Embryos grown from the sperm and eggs are then chosen to be transferred into the woman's uterus. Assisted reproductive technology in women depends on ovarian stimulation and concurrent multiple oocyte development, induced by exogenous gonadotropins.

Infertile women are often treated with gonadotropin treatments such as gonadotropin-releasing hormone (GnRH) flare protocols. Estrogen pre-treatment with concomitant growth hormone (GH) treatment is sometimes used in an effort to try and amplify intra-ovarian insulin-like growth factor-I (IGF-I) paracrine effect, which is expressed by granulosa cells and enhances gonadotropin action. However, the clinical utility of combined GH/ovarian stimulation is limited and responses are not drarmatic.

Dehydroepiandrosterone (DHEA) is secreted by the adrenal cortex, central nervous system and the ovarian theca cells and is converted in peripheral tissue to more active forms of androgen or estrogen. DHEA secretion during childhood is minimal but it increases at adrenarche and peaks around age 25, the age of maximum fertility, only to reach a nadir after age 60. There is evidence to support use of exogenous DHEA to increase ovulation stimulation in older women who respond poorly to gonadotropin treatments. First, studies demonstrate marked augmentation of serum IGF-I concentrations of oral administration of physiological DHEA. Second, DHEA is a steroid prohormone for ovarian follicular sex steroidogenesis.

Third, Casson studies have shown that concurrent oral DHEA supplementation over about two months and one or two stimulation cycles improved gonadotropin response by approximately two-fold in women who had normal follicular stimulating hormone concentrations, yet had poor response to ovarian stimulation. Frattarelli and Peterson found that cycle day 3 testosterone above 20 ng/dl was associated with higher IVF pregnancy rates (11.2% vs. 53.1%). Approximately 25 to 50 mg of DHEA is considered physiologic replacement for young females. Adverse effects are extremely uncommon at such dosages, while dosages as high as 1600 mg daily have caused significant side effects, requiring discontinuation of treatment.

The “aging ovary” represents the last frontier of human infertility treatment and is generally considered untreatable with current medical resources. The possibility that any intervention may significantly benefit the response of the aging ovary is therefore potentially revolutionary.

Androgens have been reported to have an effect on sex ratio. The sex allocation theory suggests that in mammals the gender of offspring is not only determined by chance but reflects characteristics of the mother and the specific quality of her physiologic environment at time of conception. Research data has supported such a contention. A number of sub-mammalian species have been demonstrated to exert adaptive controls over the gender of their offspring. Investigators have, therefore, suggested that mammals should have such abilities, as well. Over 40 major studies have demonstrated statistically significant atypical ratios for offspring, based on either maternal characteristics and/or environmental factors.

In animals, as well as humans, dominant female behavior has been associated with high androgen levels, which in turn, has been associated with an increased likelihood of conceiving male offspring. A proposed explanation by Grant and Irwin (2005) has been that fcollicular environments with high androgen levels attract Y-bearing spermatozoa, while follicles with low levels of androgens seek out X-chromosome bearing semen. Therefore, a theory has been that the effect of high androgen levels on sex ratio is before the implantation stage.

### BRIEF SUMMARY OF THE INVENTION

The present invention is directed to the administration of an androgen for at least about four consecutive months, to precondition ovulation induction in women. In one embodiment, the androgen is DHEA. DHEA administration may be conducted orally in patients. In conjunction with DHEA, high dose gonadotropins may be administered. Also in conjunction with DHEA, follicle stimulating hormone (FSH), norethindrone acetate. leuprolide acetate, and gonadotropin may be used to maximize ovulation induction.

In another aspect of the invention. a method of improving cumulative embryo score may comprise administering an androgen to a human female, for example, DHEA, for at least about four consecutive months followed by harvesting and fertilizing oocytes and forming embryos. Between about 50 mg and about 100 mg of DHEA may be administered to a human female per day. Moreover, a method of increasing the quantity of fertilized oocytes may comprise administering an androgen to a human female for at least about four consecutive months, harvesting and fertilizing the oocytes. Furthermore, a method of increasing the quantity of day 3 embryos from one cycle of in vitro fertilization may comprise administering an androgen for at least about four consecutive months, harvesting and fertilizing the oocytes and forming day 3 embryos.

In a further aspect, the invention relates to methods of normalizing ovarian DHEA levels by administering an androgen to a human female for at least about one month. In a still further aspect, the invention relates to increasing euploid number and rate of oocytes, by administering an androgen to a female for at least about four consecutive weeks.

In another aspect of the invention, a method of increasing the inhale fetus sex ratio may comprise raising androgen levels in a female by, for example, administering DHEA for at least about one month. The fetus and female may both be human and part of an in vitro fertilization process. The androgen level may be raised to above about 250 ng/dl, preferably above about 350 ng/dl. Further, raising androgen levels in an older female to above about 250 ng/dl, preferably above about 350 ng/dl, may decrease the likelihood of a miscarriage.

### BRIEF DESCRIPTION OF THE DRAWINGS

FIG. 1 is a table showing improved ovulation induction with DHEA.

FIG. 2 is a graph showing increase in the number of fertilized oocytes resulting from oocytes harvested from women with DHEA treatment.

FIG. 3 is a graph showing increase in the number fertilized oocytes resulting from oocytes harvested from women with at least 4 weeks of DHEA treatment.

FIG. 4 is a graph showing an increase in the number of day three embryos resulting from oocytes harvested from women with at least 4 weeks of DHEA treatment.

FIG. 5 is a chart showing chemical pathways of adrenal function.

### DETAILED DESCRIPTION OF THE INVENTION

When attempting in vitro fertilization, older women produce few oocytes and yield few normal embryos, even when exposed to maximal gonadotropin stimulation. The decreased ability of older women to respond to ovulation inducing medications is evidence that ovarian reserve declines with age. The ability of women to respond to ovulation inducing medications declines with age. With IVF cycles, older women produce few oocytes and yield few normal embryos when exposed to maximal gonadotropin stimulation. This change in ovarian responsiveness is known as diminished ovarian reserve.

The reservoir of primordial follicles is steadily depleted throughout life. The transition from primary follicle to pre-antral follicle can take between about four and about six months. On the way, atresia and apoptosis are responsible for the disappearance of most follicles that have initially been recruited in a cycle. As follicles grow they move through the stages of primary, preantral, antral, and preovulatory follicles and finally to ovulation. The majority of follicles end in hormonally controlled apoptosis known as atresia, only a few ever mature to ovulation. A change in follicular dynamics with improved survival of follicles to the early antral stages, when gonadotropin dependent cyclic recruitment can influence further follicular growth may be one potential mechanism by which DHEA affects oocyte quantity and quality.

Androgens can influence ovarian follicular growth either by acting as metabolic precursors for steroid production, as ligands for androgen receptors or by some non-classical mechanism. Adrenal androgen and androgens produced by the theca cell act as prehormones for granulose cell production of estradiol. Human granulose cells are a site of sulfatase activity and dehydroepiandrosterone-sulfatase (DHEAS) and DHEA can be utilized as a substrate for androstenedione and estrogen production. During ovulation induction with exogenous gonadotropins DHEA is the prehormone for up to about 48% of follicular fluid testosterone, which is itself the prehormone for estradiol.

Androgens act together with FSH to stimulate follicular differentiation. IGF-1 expression is higher in preantral and early antral follicles of DHEA treated rat ovaries. In vitro cultured murine preantral follicles increase follicle size and DNA synthesis in response to androgens but not to estrogens. Preantral murine follicles are unresponsive to recombinant human follicle stimulating hormone (rFSH) but respond synergistically to combinations of androgen and rFSH. Mice lacking androgen receptor have impaired fertility and evidence of defective early folliculogenesis. In rodents androgens enhance recruitment of primordial follicles into the growth pool but cause atresia of late antral follicles.

The potent androgen receptor agonist dihydrotestosterone (DHT) stimulates proliferation of porcine granulose cells and inhibits progesterone production. The action of DHT in porcine follicles was greater in about 1 mm to about 3 mm follicles than in about 3 mm to about 5 mm diameter follicles. Androgens are known to promote steroidogenesis, follicular recruitment and to increase insulin like growth factor in the primate ovary. Androgen receptors are absent in human primordial and primary, follicles but there is evidence of nuclear staining for androgen receptor in human granulose and thecal cells in secondary follicles.

Improved IVF outcomes are reported among women with higher baseline testosterone levels. Higher serum testosterone is correlated with higher estradiol and oocyte numbers retrieved for IVF. Improved outcomes in women with diminished ovarian reserve after co-treatment with an aromatase inhibitor during cycle stimulation may be the consequence of FSH induction. The resultant ovarian response then leads to improved follicular survival, increased follicle numbers and higher estradiol levels during stimulation, as also observed in polycystic ovarian disease.

It is possible that DHEA treatment may create polycystic ovaries (PCO)-like characteristics in the aging ovary. Human PCO's have been described as representing a “stock-piling” of primary follicles secondary to an alteration at the transition from primordial to primary follicle. Possible mechanisms suggested for this observation are abnormal growth factors, increased LH, or increased ovarian androgen. Normal ovarian theca cells of the pre-antral follicle produce androstenedione, DHEA, and testosterone. Women with polycystic ovaries have higher serum testosterone, androstenedione and DHEA compared to controls and higher ovarian venous levels of DHEA, androsterone and testosterone. Long term exogenous androgen exposure can induce PCO like histological and sonographic changes in normal ovaries similar to PCO.

DHEA may be administered to a human female at a dose of between about 50 mg/day and about 100 mg/day, preferably between about 60 mg/day and about 80 mg/day, and in one study about 75 mg/day. DHEA may have an effect on women after about 4 weeks, but the effect may increase over time. DHEA effects may reach statistically significant effects after about 4 months of use, but may continue to increase past four months of use. In one study ovulation induction was accomplished with norethindrone acetate, leuprolide acetate, human menopausal gonadotropin, and follicle-stimulating hormone.

A DHEA dose of about 1600 mg daily may result in significant adverse effects, often requiring the discontinuation of the medication. The safety issue of most concern is that DHEA—as a precursor of sex steroids—may increase the risk of estrogen- or androgen-dependent malignancies. Pregnancy, in itself is a high a androgen state, and women with polycystic ovarian diseases, also a high androgen state, do not generally deliver daughters with masculinized external genitalia. This suggests that the limited and low-dosage use of DHEA in infertility patients should be safe. DHEA is currently available in the U.S. without prescription.

Possible side effects associated with DHEA use are acne, deepening voice and facial hair growth, though long-term effects of DHEA administration are unknown. As a precursor of sex steroids one, of course, has to be concerned abut the potential effect on hormone-sensitive malignancies.

I. Improvements in Ovulation

Treatmnents with an androgen, alone or in conjunction with other hormones, increase a woman's response to ovulation induction, measured in both oocyte and embryo yield. Androgens may be, for example, dehydroepiandrosterone (DHEA) or testosterone. DHEA treatment is an adjunct to ovulation induction. DHEA taken orally for at least about one month, preferably for about four months, before initiating gonadotropin treatment may prepare the ovaries for gonadotropin stimulation. It is believed that a larger response may be obtainable by combining gonadotropins and DHEA in treatment over an at least about four month period before an IVF cycle.

Young ovaries are characterized by large numbers of antral follicles and a low rate of atresia. In contrast, older ovaries have few antral follicles, high rates of atresia and exhibit increasing “resistance” to ovulation induction. With IVF, older women have decreased oocyte quantity and quality, produce fewer high quality embryos and have lower implantation and pregnancy rates. Most follicular atresia occurs after the primordial follicle resumes growth but before it is gonadotropin responsive enough for recruitment. An induced delay in onset of atresia may salvage follicles for possible ovulation. Interestingly, such an “arrest” of the atretic process has been noted among anovulatory women with polycystic ovary syndrome (PCO). For these women follicles remain steroidogenicaly competent and show evidence of increased aromatase activity compared to like-sized follicles from normal ovaries. Follicular hypersecretion of DHEA, which is typical of PCO, is associated with increased aromatase activity. The increased yield of oocytes and embryos experienced by patients undergoing DHEA treatment also suggest this underlying physiological process.

II. Improvements to Cumulative Embryo Score

DHEA use may have a beneficial effect on oocyte and embryo quality. The observation that DHEA treatment was associated with improved cumulative embryo scores may infer that such treatment leads to improved embryo and egg quality. This suggestion is further supported by strong trends towards improved euploidy in embryos and improved pregnancy rates.

Cumulative embryo score is determined by multiplying the number of cells in the embryo by the embryo grade. Embryo grade is a judgment of the embryologist on embryo quality from 1 to 5. Most good embryos are scored 4, with 5 reserved for exceptional embryos. The grade is based on the uniformity of the cells, the color and consistency of the cytoplasm, and the amount of fragmentation. Normal embryos are less than 5% fragmented. A woman with three eight cell embryos each with a grade of four would have a cumulative embryos score of 96, the product of 3×8×4.

A cumulative embryo score for women prior to DHEA use may have been about 34. A cumulative embryo score after DHEA use of at least about four consecutive months may be at least about 90, preferably at least about 95, and in one study at least about 98. The increase in cumulative embryo score may be at least about 56, preferably at least about 60, and in one study about 64. The difference in the cumulative embryo score prior to DHEA use and the cumulative embryo score after DHEA use may be statistically significant, p<0.001.

III. Increase in the Number of Fertilized Oocytes

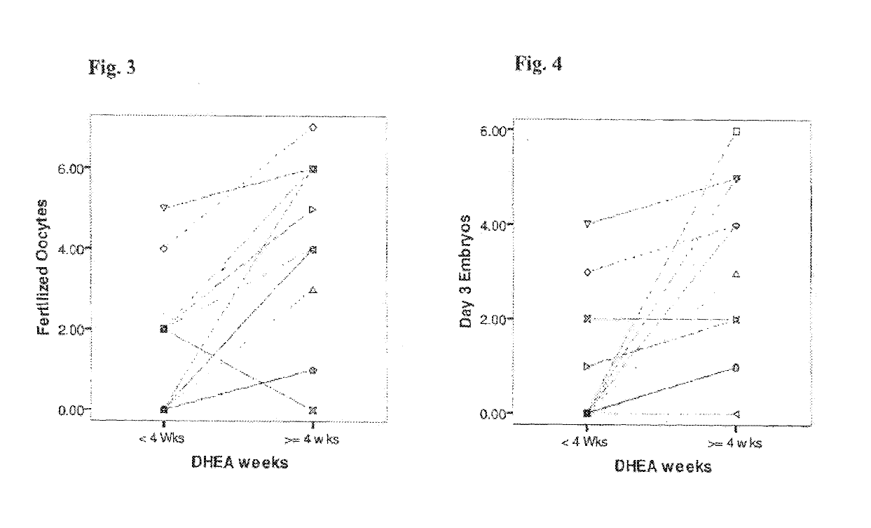

The number of fertilized oocytes produced by women significantly increased after at least about 4 months of consecutive DHEA treatment in 12 women, even though slight improvements were shown after at least about four weeks of consecutive DHEA treatment, as shown in FIG. 3. As shown in FIG. 3, paired comparisons of fertilized oocytes from women having less than about four consecutive weeks of DHEA treatment to the same women having at least about four consecutive weeks of DHEA treatment showed an increase of about 2 fertilized oocytes, or a median increase of about 2.5 fertilized oocytes. The number of fertilized oocytes may show more significant increase after at least about 4 months of DHEA treatment, and may show maximal increase after at least about eight months of DHEA treatment,

IV. Increase in the Number of Day 3 Embryos

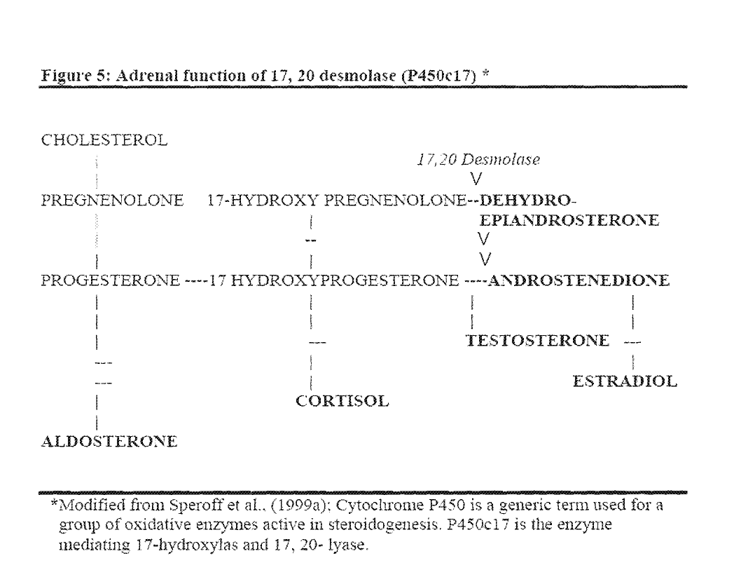

The number of day 3 embryos produced by women also may significantly increase after at least about four months of consecutive DHEA treatment in 12 women, even though slight increases may be shown after at least about 4 weeks of DHEA treatment, as shown in FIG. 4. All of the day 3 embryos included in the study were normal based on their appearance and on the number of cells, i.e. at least four cells. Paired comparisons of fertilized oocytes from women having less than about four consecutive weeks of DHEA treatment to the same women having at least about four consecutive weeks of DHEA treatment may show an increase of about 1 day 3 embryo, and in the study summarized in FIG. 4, an increase of about 2 day 3 embryos. While the number of day 3 embryos produced slightly increased after at least 4 weeks of DHEA treatment, more significant increase occurs after at least about 4 months of DHEA treatment, and maximal increase may occur after at least about eight months of DHEA treatment.

V. Increase in the Number of Euploid Oocytes

DHEA may improve the number of euploid embryos and embryo transfers in women with diminished ovarian reserve (DOR). Pretreatment with DHEA, for at least about one month, preferably at least about four months, in women may increase oocyte and embryo quantity, egg and embryo quality, cumulative pregnancy rates, pregnancy rates with IVF and time to pregnancy. We evaluated the prevalence of aneuploidy in embryos, produced through IVF, from 27 consecutive IVF cycles in women with DOR who also had undergone preimplantation genetic diagnosis (PGD). Amongst those, 19 had entered IVF without DHEA treatment and eight had received DHEA supplementation for at least four weeks prior to IVF start.

DHEA treatment may result in higher oocyte numbers (10.4±7.3 vs. 8.5±4.6). A significantly larger number of DHEA treated IVF cycles (eight out of eight, 100%) had at least one euploid embryo for transfer than in untreated cycles (10/19, 52.6%; Likelihood ratio, p=0.004; Fisher's Exact Test, p=0.026). Neither absolute numbers of euploid embryos after DHEA, nor percentages of embryo ploidies differed, however, significantly between untreated and treated patients.

Thus, pretreatment with DHEA of women with DOR may significantly increase their chances for the transfer of at least one euploid embryo.

VI. Improvements to Ovarian Function

As shown in FIG. 5, the adrenal enzyme 17,20-desmolase may be responsible for the conversion of 17-hydroxy pregnelonone into DHEA (and the conversion of 17-hydroxyprogesterone into androstenedione) which, based on the two-cell two-gonadotropin theory, may serve in the ovary as a precursor substrate for estradiol and androgens. DHEA substitution may rejuvenate certain aspects of ovarian function in older ovaries. Since DHEA declines with age to a very significant degree, intraovarian DHEA deficiency may be causally related to the ovarian aging process.

DHEA may have beneficial effects on ovarian function, and oocyte and embryo quality. How DHEA exerts these effects on the female ovary has remained open to speculation. Casson et al suggested that it may occur through an increase in insulin-like growth factor-I within the intraovarian environment (Casson et al., 1998). Others have suggested that the intraovarian increase in androgens, by itself, may improve ovarian response to stimulation, possibly by improving the sensitivity of FSH receptors on granulose cells. (Garcia-Velasco et al., 2005). We have suggested that, based on the two-cell, two-gonadotropin model (Hillier et al., 1994), DHEA serves as substrate for the production of estradiol. Since DHEA significantly declines with age (Speroff et al., 1999), this substrate decreases, resulting in lower estradiol (and androgen) levels after ovarian stimulation with gonadotropins (Barad and Gleicher. 2005 and 2005a). DHEA substitution would then be expected to reverse the deficiency in substrate and, therefore, increase estradiol (and androgen) levels.

FIG. 5 shows the pathways for normal adrenal function. A patient with abnormal 17,20 desmolase (P450c17) function may have a hormone profile characterized by persistently low DHEA, androstenedione, testosterone and estradiol levels, but normal aldosterone and cortisol levels. The patient exhibited some of the classical signs of prematurely aging ovaries (Nikolaou and Templeton, 2003; Gleicher N, 2004) which include ovarian resistance to stimulation, poor egg and embryo quality and prematurely elevated FSH levels.

We have previously suggested that the decrease in DHEA levels, with advancing female age, may be an inherent part of the ovarian aging process and may, at least in part, and on a temporary basis, be reversed by external DHEA substitution (Barad and CGleicher, 2005). T his case demonstrates that low DHEA levels are, indeed, associated with all the classical signs of (prematurely) aging ovaries. While association does not necessarily suggest causation, the observed sequence of events in this patient supports the notion that low DHEA levels adversely affect ovarian function.

The patient was initially thought to have largely unexplained infertility. Approximately 10 percent of the female population is believed to suffer from premature aging ovaries and this diagnosis is often mistaken for unexplained infertility (Nikolaou and Templeton, 2003, Gleicher N, 2005). The patient later developed signs of prematurely aging ovaries and, finally, even showed elevated FSH levels. In the time sequence, in which all of these observations were made, the patient followed the classical parallel, premature aging curve (Nikolaou and Templeton, 2003; Gleicher N, 2005).

Once substituted with oral DHEA a reversal of many findings characteristic of the aging ovary was noted. First, the patient's DHEA and DHEAS levels normnalized. In subsequent natural cycles an apparently normal spontaneous follicular response was observed, with normal ovulatory estradiol levels in a patient with persistently low estradiol levels before DHEA treatment (Table 2). The response to ovarian stimulation improved, quantitatively and qualitatively, as the patient improved peak estradiol levels, oocyte and embryo numbers and, as the successful pregnancy may suggest, also embryo quality.

DHEA deficiency may be a cause of female infertility and may be a possible causative agent in the aging processes of the ovary. It also presents further confirmation of the value of DHEA substitution whenever the suspicion exists that ovaries may be lacking of DHEA substrate. Since the process is familial (.Nikolaou and Templeton, 2003), it is reasonable to assume that, like other adrenal enzymatic defects, 17,20-desmolase deficiency, may occur either in sporadic or in an inherited form. As both forms will result in abnormally low DHEA levels, both may lead to phenotypical expression as premature ovarian aging.

That there may be a genetic components to the aging process of ovaries has also been suggested by recent observations of IVF outcomes in different racial groups which offer evidence that the physiological aging curves in African American and Asian, in comparison to Caucasian, women may be shifted towards younger age (Grainger et al., 2004; Purcell et al., 2004; Gleicher and Barad, 2005).

VII. Increase in Spontaneous Conceptions

After DHEA treatment, there may be an unexpectedly large number of spontaneous conceptions in women waiting to go into an IVF cycle. The DHEA treatment may be at least about 2 weeks before spontaneous conception occurs. In the population of women who are waiting to go into IVF, the spontaneous pregnancy rate is a fraction of 1% per month. However, in the population of women who have been on DHEA treatment, there were 13 spontaneous pregnancies out of 60 women, or about 22%. This may provide evidence that DHEA works not only in conjunction with gonadotropin stimulation of ovaries, but also without gonadotropin stimulation of ovaries.

VIII. Increase in Male Fetus Sex Ratio

Raising androgen levels in a female may increase the male fetus sex ratio. The gender of offspring may not be solely determined by chance. Higher androgenized female mammals give birth to more male offspring. Androgens, such as DHEA, may be utilized and an elevated baseline level of above about 250 ng/dl, preferably above about 350 ng/dl, may be sufficient. Treated infertile women with diminished ovarian reserve long-term with DHEA established a human model to investigate this theory. Data obtained from this model support a possible effect of androgenization on gender not through a follicular selection mechanism but rather through different mechanisms than previously theorized as evidenced by occurring after the preimplantation embryo stage.

Our routine treatment protocol involves 25 mg of micronized, pharmaceutical grade DHEA, TID, which will uniformly raise levels of unconjugated DHEA above 350 ng/dl and, therefore, raise baseline testosterone. In the six pregnancies, spontaneously conceived, the distribution between female and male offspring was equal, at three and three, respectively. Whereas amongst the remaining 15 offspring, which were products of pregnancies achieved through IVF, the distribution was 12 males and 3 females (p=0.035). Amongst women undergoing IVF and PGD, 53 embryos were analyzed from 17 IVF cycles, all having undergone ICSI. The gender distribution was not significantly skewed, with 27 being male and 26 female.

The data, demonstrating a strong trend towards significance overall, and significance (p=0.035) amongst IVF patients, suggest that gender determination may be influenced through hormone environments. The even distribution of gender in this group of patients argues against a selection process towards male, which is driven by the follicular environment, as has been previously suggested. The even distribution of gender in preimplantation embryos, seen in the control group, also speaks against such an effect.

The only remaining conclusion from the here presented data is that female androgenization affects gender selection after the preimplantation embryo stage and that, by definition, identifies the stage of androgenic influence on gender at or after implantation. All, but one, IVF cycles in study and control groups underwent ICSI, which requires the removal of granulose cells from the oocyte. One hypothesis is that such a removal may render the local environment more favorable towards the implantation of male than female embryos. A second hypothesis would suggest a similar effect, based on the difference in hormonal milieu in the luteal phase between IVF and spontaneous conception cycles, with the former uniformly supported by progesterone and the latter only sporadically, or not at all. The data provides evidence that the androgenization of females may increase the prevalence of male offspring, especially with IVF.

### EXAMPLE 1

### Improved Ovulation

A 43 year old woman undergoing IVF with banking of multiple cryopreserved embryos for future aneuploidy screen and transfer is administered an androgen, namely DHEA. In ten months she undergoes eight treatment stimulation cycles while continuously improving her ovarian response, resulting in oocyte and embryo yields far beyond those previously seen in a woman her age.

The patient's history is unremarkable except for two previous malarial infections. She is allergic to sulfa medications and has a history of environmental allergies. Her surgical history includes umbilical hernia repair at age one and cholecystectorny at age 21. She had used oral contraceptives for over 10 years. She has no history of irregular menstrual cycles.

Day three serum FSH and estradiol (E2) in her first IVF cycle are 11 mIU/ml and 18 pg/ml, respectively. In subsequent cycles her baseline FSH is as high as 15 mIU/ml. She is given an ovulation induction protocol which is prescribed for patients with evidence of decreased ovarian reserve. Briefly, the protocol includes the following medications: norethindrone acetate tablets (10 mg) for 10 days, starting on day two of menses, followed three days later by a “microdose” dosage of 40 μg of leuprolide acetate, twice daily, and, after another three days, by 600 IU of FSH (Gonal-F; Ares-Serono, Geneva, Switzerland) daily. Peak serum E2 concentration on day nine of stimulation is 330 pg/nml. Following, injection of 10,000 IU human chronic gonadotropin (hCG), she undergoes oocyte retrieval. Only one oocyte is obtained and one embryo is cryopreserved.

Because of the poor response to ovulation stimulation, she is advised to consider donor oocyte or embryo donation. She rejects both options. She starts a second cycle using the same stimulation protocol with one exception: instead of 600 IU of FHS daily, the patient received 450 IU of FSH and 150 IU of human menopausal gonadotropin (HMG, Pergonal, Ares-Serono, Geneva, Switzerland). This stimulation protocol is continued in identical fashion for the remaining cycles. However, two weeks before starting her second cycle, she begins administration of 75 mg per day of oral micronized DHEA. The date on which she begins administration of 75 mg per day of oral micronized DHEA is Oct. 6, 2003.

Methods

The eight treatment cycles are divided into three groups to allow statistical comparison: pre-initiation and very early use of DHEA (early=cycles 1 and 2), initial cycles (mid=cycles 3 -5), and later cycles (late=cycles 6 -8). Comparison between these categories is by one-way analysis of variance (ANOVA) and multiple comparisons by Student-Neuman-Keuls (SNK) test. The homogeneity of variances and used orthogonal linear contrasts are tested to compare groups and polynomial contrast to test for linear and quadratic trends. All outcomes are presented as mean ±1 standard deviation. Rate of change of oocyte counts, cryopreserved embryos and (log transformed) peak estradiol between subsequent cycles is estimated by linear regression.

Embryos are evaluated by the embryologists on day three post-insemination for cell-count and grading. Embryo grading is based on a 1 to 4 scale depending on symmetry, percent fragmentation and appearance of the cytoplasm. All viable embyros are cryopreserved. Statistics are performed using SPSS for Windows, Standard version 10.0.7 (SPSS Co., Chicago, Ill.). Assay of E2 and FSH are performed using the ACS: 180 chemoluminescence system (Bayer Health Care LLC, Tarrytown, N.Y.).

A method of preconditioning ovulation induction in a human female is conceived, comprising administering an androgen in a female for at least about four consecutive months. In one embodiment, the androgen is DHEA. Administration of DHEA for at least about four consecutive months may further comprise administering high dose gonadotropins to the female. Furthermore, DHEA may be administered along with follicle stimulating hormone, human menopausal gonadotropin, norethindrone acetate, leuprolide acetate, and human chronic gonadotropin. DHEA may be administered orally.

The length of time the androgen is administered to the female can be at least four consecutive months. The DHEA treatment may continue for more than four months. In one embodiment, the androgen administered is DHEA.

Results

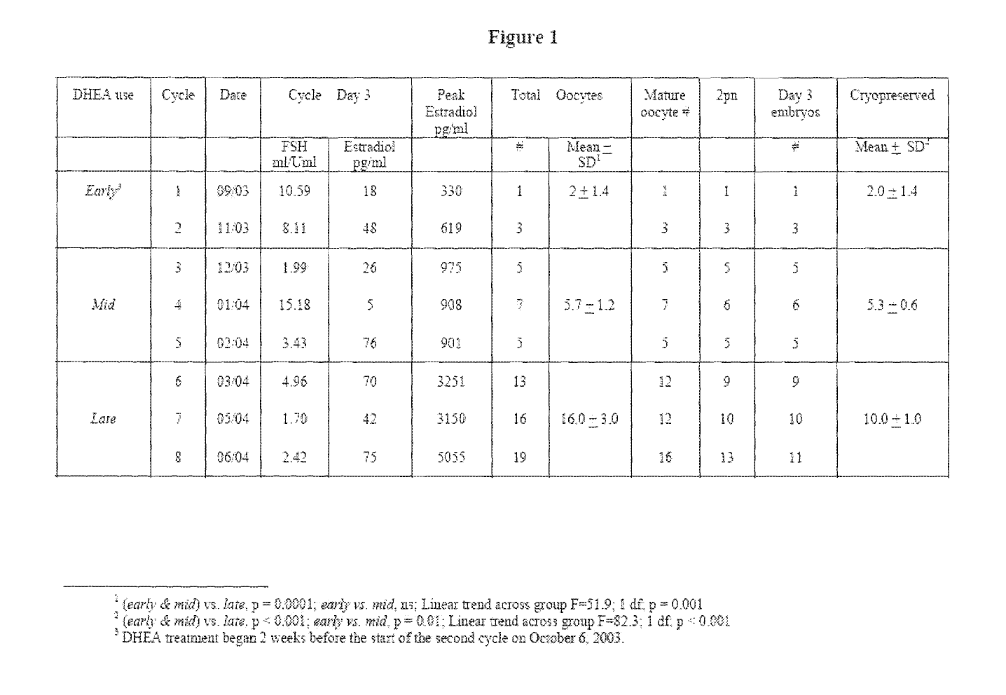

The results of ovulation induction are displayed in FIG. 1. After eight stimulation cycles and approximately eight months of DHEA treatment, the patient produced 19 oocytes and 11 cryopreservable embryos. A total of 50 viable embryos have so far been cryopreserved. Significantly more oocytes (p=0.001) and cryopreserved embryos (p<0.001) are obtained in the late cycles (cycles 6-8, 4+ consecutive months of DHEA treatment) compared to the combined early and mid cycles (cycles 1-5, 0-4 consecutive months of DHEA treatment). There is no significant difference in average embryo cell count (6.83±1.37 vs. 7.2±1.15) or morphology (3.6±0.5 vs. 3.7±0.5) between early and mid compared to late cycles. Peak E2, total oocyte, and embryos cryopreserved increase linearly from cycle to cycle, as shown in FIG. 1. Oocyte yield increase 2.5±0.34 oocytes per cycle (p<0.001), cryopreservable embryo yield increase 1.4±0.14 embryos per cycle (p<0.001) and (log) peak E2 increase 0.47±0.06 (p<0.001) across treatment cycles.

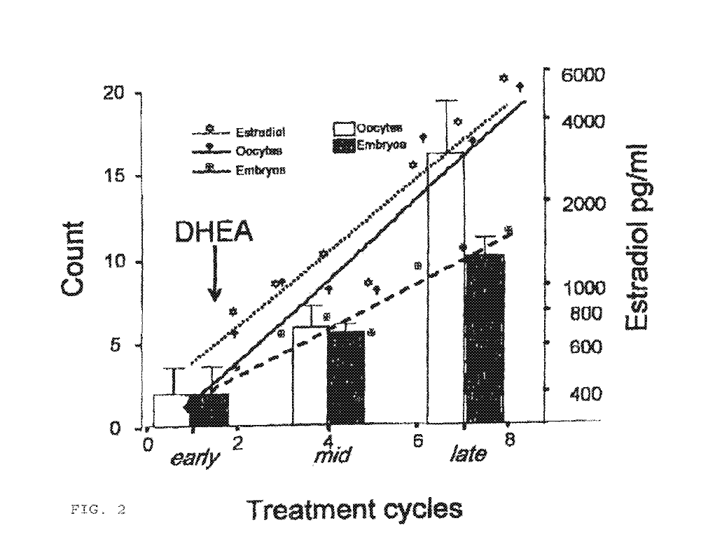

The linear increase in (log) peak E2 shown in FIG. 2 represents a cycle to cycle rate of increase from 123 pg/ml/cycle to 1491 pg/ml/cycle over the eight cycles of treatment. After adjusting for cycle day, the (harmonic) mean E2 is 267 pg/ml (95% confidence intervals (CI) 143 to 498 pg/ml) in the early phase, 941 pg/ml (95% CI 518 to 1712 pg/ml) in the mid phase, and 1780 pg/ml (95% CI 1121 to 2827 pg/ml) in the late phase of treatment. Each of these homogeneous subsets is significantly different from the other (p<0.05) by SNK multiple comparison testing.

The dramatic increase in oocyte and embryo yield experienced by this 43 year old woman is completely surprising and unexpected. The patient's post-DHEA response to ovulation induction has become more like that of a younger woman with PCO, than that of a 43 year old woman. Since starting DHEA treatment, the patient has produced 49 embryos of high enough quality to undergo cryopreservation. Sixty percent of those embryos were produced in the last three cycles of treatment, which took place after at least about four consecutive months after starting treatment. After producing only one embryo prior to starting DHEA treatment, the patient improved by an order of magnitude and produced 13 oocytes and 9 embryos in a cycle after at least about four consecutive months of DHEA treatment, 16 oocytes and 10 embryos in a cycle after at least about five and a half consecutive months of DHEA treatment, and 19 oocytes and 11 embryos in a cycle after at least about seven consecutive months of DHEA treatment.

The increasing numbers of cryopreservable embryos may suggest that embryo quality has improved. Quantity of embryos definitely is improved and quality may be improved. This patient also took high dose gonadotropins along with DHEA for several months.

The preceding example is to be construed as merely illustrative and not limitative of the remainder of the disclosure in any way.

### EXAMPLE 2

### Improved Oocyte Fertilization and Cumulative Embryo Score

Thirty (30) patients with evidence of decreased ovarian reserve were given supplemental DHEA 25 mg three times a day, for a total of 75 mg per day, for an average of about 4 months before beginning ovulation induction for IVF. Twelve patients contributed data from cycles both pre-DHEA and post-DHEA, eleven patients contributed data from cycles only pre-DHEA, and seven patients contributed data from cycles only post-DHEA. Patients' response to ovulation induction before DHEA treatment was compared to patients' response to ovulation induction after DHEA treatment with regard to peak estradiol, oocyte production, and embryos transferred and embryo quality.

The thirty patients contributed to data for 42 total cycles, 23 cycles prior to and 19 cycles after starting DHEA supplementation. In comparing the patients as a group pre- and post-DHEA treatment cycles, there were improvements in cancellation rate, peak estradiol, average day 3 embryo cell counts, and embryo grade, but the improvements were not statistically significant. However, average oocyte numbers, eggs fertilized, day-three embryos, embryos transferred and cumulative embryo scores increased significantly after DHEA treatment. In logistic regression models adjusted for oocyte number, there was evidence of improved fertilization rates (p<0.005), increased numbers of day-three embryos (p<0.05), and of improved overall embryo score (p<0.01). In 34 IVF cycles that reached the embryo transfer stage, a positive pregnancy test was obtained in zero of 16 cycles with less than an average of about 4 months of DHEA treatment and in 4/18 (22%) cycles after an average of 4 months of DHEA treatment.

This case series illustrates the possibility that some ovarian function can be salvaged, even in women of advanced reproductive age.

TABLE 1
Univariate comparison of results of in vitro fertililization
before and after treatment with DHEA.

| | Pre DHEA | Post DHEA | p |
|---|---|---|---|
| N | 23 | 19 | |
| Age | 40.9 ± 0.7 | 42.8 ± 0.7 | ns |
| Weeks of DHEA | — | 16.1 ± 2.4 | — |
| Cancellation | 5/21 (21%) | 1/19 (5%) | ns |
| Peak Estradiol | 1018 ± 160 | 1192 ± 904 | ns |
| Oocytes | 3.3 ± 0.7 | 5.8 ± 1.0 | 0.04 |
| Fertilized eggs | 1.3 ± 0.3 | 4.6 ± 0.8 | <0.001 |
| Average Day 3 | 3.1 ± 0.6 | 4.5 ± 0.5 | ns |
| embryo cell count | | | |
| Average Day 3 | 2.4 ± 0.3 | 2.8 ± 0.3 | ns |
| embryo grade | | | |
| Cumulative | 34 ± 6.8 | 98 ± 17.5 | 0.001 |
| Embryo Score | | | |
| Transferred embryos | 1.0 ± 0.2 | 2.6 ± 0.4 | 0.001 |
| Number of Day 3 | 0.9 ± 0.2 | 3.2 ± 0.6 | 0.001 |
| embryos | | | |
| Positive hCG | 0/16 | 4/18 | ns |
| (per transfer cycle) | | | |

Cycle characteristics and responses to treatment are shown in Table 1. The average age of the patients who began DHEA was 41.6±0.6 years. Women in the DHEA group used DHEA for a median value of 16 weeks before their IVF cycle. The cycle cancellation rate was 5 of 21 cycles (21%) pre-DHEA and 1 of 19 (5%) post-DHEA. There was no statistically significant difference in peak estradiol levels between pre- and post-DHEA cycles.

Continuing with the cycle outcomes presented in Table 1, there are improvements in average cell count of day-three embryos and mean embryo grade after DHEA treatment, however the differences are not significant. Mean oocyte numbers, fertilized eggs, day-three embryos, embryos transferred and cumulative embryo scores, all increased significantly alter DEHA treatment. In the models adjusted for oocyte number, there was still evidence of increased fertilization rates (1.93 fertilized oocytes, 95% C.I. 0.82-3.04; p<0.005), increased numbers of day-three embryos (1.36 embryos, 95% C.I. 0.34-2.4; p<0.05), and of improved overall embryo score (32.8, 95% C.I. 9.6-56; p<0.01).

FIG. 3 shows paired comparisons of fertilized oocytes (average increase 2.5±0.60; p=0.002) among 12 patients with DHEA treatment cycles of less than about 4 weeks to fertilized oocytes in the same 12 patients after at least about 4 weeks of DHEA treatment. FIG. 4 shows paired comparisons of day 3 embryos (average increase 2.0±0.57; p=0.005) among 12 patients with DHEA treatment cycles of less than about 4 weeks and at least about 4 weeks during IVF cycles. The paired comparisons shows that the mean increase in the number of fertilized oocytes was modest, but significant, (1.42±0.63 increased numbers of fertilized oocytes; p<0.05).

The mean increase in embryo scores was 57±14.7 (p<0.01). The increase in the number of day 3 embryos was 2.0±0.57 (p=0.005) (See FIG. 4) and the increased fertilization quantity was 2.5±0.60 fertilized oocytes per patient (p=0.002) (See FIG. 3).

In addition, two patients achieved ongoing pregnancies while taking DHEA without IVF; one (43 year old) while using DHEA during a stimulated IUI (intrauterine insemination) cycle and a second (37 year old) conceived spontaneously following an unsuccessful IVF cycle. A third patient (40 year old) also conceived spontaneously while preparing for an IVF cycle; however that pregnancy ended in a spontaneous abortion. In all 7 of 45 (16%) patients using DHEA have conceived and 5 of 45 patients (11%) have experienced continuing pregnancies.

### EXAMPLE 3

### Increased Euploidy Rate

In another study (data not shown), patients were analyzed after four weeks of DHEA treatment. Seven patients had embryos tested by pre-implantation genetic diagnosis (PGD). In three women who had PGD after less than four weeks of DHEA usage and a mean age 41.5±5.1 at the time of starting IVF cycles, the euploidy, or normal chromosome number, rate was 2/30 embryos (6.6%). In six patients who had PGD after more than four weeks of DHEA usage, and a mean age of 43.7±1.3 years at the time of starting IVF cycles, the euploidy rate increased to (8/27; 29.6%), though this trend did not reach statistical significance. There is a mean age difference between patients who underwent IVF after less than four weeks of DHEA usage (mean age 41.5±5.1) and patients who underwent IVF after at least four weeks of DHEA usage (mean age 43.7±1.3).

As women age, there is a substantial decline in euploidy rates in embryos produced. Thus, the increase in euploidy results in older women is dramatic evidence of the effectiveness of DHEA in improving embryo quality because even an identical euploidy result between older women and younger women would indicate effectiveness of DHEA.

### EXAMPLE 4

### DHEA Treatment Increases Euploidy Number

In a series of studies we have documented that DHEA supplementation in women with diminished ovarian reserve (DOR) increases egg and embryo count, improves egg and embryo quality, increases pregnancy rates and shortens time to conception. An improved ovarian response to stimulation in women with DOR was also reported by Casson et al. Improved pregnancy rates with higher androgen levels and improved embryo quality after androgen priming with aromatase inhibitors have also been reported.

All of these reports point towards improvements in follicular recruitment after treatment with androgenic compounds in both, a quantitative and a qualitative sense, even though the potential physiologic mechanisms leading to such an effect are still not well understood. Some authorities have suggested that higher testosterone levels sensitize granulosa cells to the stimulator effects of follicle stimulating hormone. Since DHEA effects peak only after approximately four months, and since this time period is approximately reflective of the full follicular recruitment cycle, we concluded that DHEA may, at least in part, affect follicular recruitment processes, possibly by influencing apoptosis. Androgens have been reported to affect granulosa cell apoptosis.

While women with prematurely DOR appear to have normal embryonic aneuploidy rates, older women, with physiologic aging ovaries, demonstrate very high aneuploidy rates of their embryos. Increasing aneuploidy rates with advancing female age are, therefore, considered a primary cause for diminishing pregnancy chances, and an increasing miscarriage risk, in older women. Since treatment with androgenic compounds in such patients appears to improve embryo quality and pregnancy chances, these observations raise the question whether such treatment may not also positively affect the prevalence of aneuploidy rates and, therefore, the availability of euploid embryos for conception. The here presented study was designed to offer a preliminary answer to this question.

Materials and Methods

We retroactively reviewed all IVF cycles performed at our center between 2004 and 2006 for cycles performed in women with a diagnosis of DOR. The study population, involving 27 IVF cycles, was then selected amongst those cycles which, in addition, had undergone preimplantation genetic diagnosis (PGD), though, in order to preclude selection biases due to underlying reasons for the performance of PGD, only for the indications of advanced female age and elective gender selection.

The diagnosis of DOR was made based on abnormally high, age stratified baseline FSH levels, as previously reported. In practical terms this meant that a diagnosis of DOR was reached if baseline FSH levels exceeded the 95% confidence interval of age appropriate levels, independent of prior IVF retrieval and/or oocyte numbers. At, or above age 43, all patients were considered to suffer from DOR, independent of baseline FSH level.

Since the year 2004, women with proven DOR, who had undergone at least one prior ovarian stimulation, demonstrating ovarian resistance based on inadequately low oocyte numbers, routinely were offered oral DHEA supplementation (25 mg TID) prior to any further IVF cycle starts. If under age 40, DHEA was given for up to four months prior to IVF. Women of older age received DHEA, if possible, for at least two months.

Women with DOR, who had no proof of ovarian resistance, were not placed on DHEA supplementation until such proof was obtained, unless they were at, or above, age 43 years. IVF cycles on DHEA supplementation have, therefore, to be considered as more severely affected by DOR than those cycles that were conducted without such supplementation. This fact is also reflected by the baseline cycle characteristics of DHEA-treated, and -untreated, patients (Table 2), which demonstrate trends towards older age and higher baseline FSH levels in DHEA treated patients.

TABLE 2
Baseline characteristics of DHEA-treated, and -untreated, patients¹

| | DHEA-TREATED | DHEA-UNTREATED |
|---|---|---|
| | n = 8 | n = 19 |
| Age (± SD, year) | 41.2 ± 4.7 | 38.9 ± 5.1 |
| Baseline FSH² ± SD | 12.4 ± 9.2 | 9.0 ± 2.7 |
| (mIU/ml) | | |
| Baseline Estradiol² ± SD | 59.7 ± 32.2 | 68.1 ± 59.1 |
| (pg/ml) | | |

¹None of the baseline parameters, listed in the table, differed to a statistically significant degree between the two groups.
²Reflects highest baseline level of each patient, and not necessarily the baseline level of the IVF cycle.

For the purpose of this analysis, a patient had to be for at least one month (30 days) on DHEA supplementation in order for the IVF cycle to be considered amongst DHEA—treated cycles. All other DOR patients were considered to have received no DHEA treatment. Following this definition, 19 DOR patients had received no DHEA supplementation, and eight had.

All women with DOR, independent of DHEA supplementation, were stimulated with identical protocols, as previously reported in detail elsewhere. In short, they, without exception, received a microdose agonist protocol with maximal goandotropin stimulation of 600 IU to 750 IU daily, with preponderance of FSH, and a smaller daily amount of human menopausal gonadotropin (hMG).

PGD was performed in routine fashion, as also previously described in detail, and involved the analysis of chromosomes X, Y, 13, 16, 18, 21 and 22 by fluorescence in situ hybridization (FISH) on day three after fertilization. Embryo transfer occurred on day five after fertilization.

Patients were represented by only one cycle outcome in each group. If patients had undergone more than one cycle, either with, or without, DHEA supplementation, only their latest cycle was included in the analysis. Three patients underwent both a pre-DHEA and a post-DHEA cycle and in those cases both cycles were included in the analysis.

Statistical analysis was performed using SPSS for windows, standard version 10.0.7. Data are presented as mean ± one standard deviation, unless otherwise noted, and statistical differences between the two study groups were tested by Chi-square and (two-sided) Fisher's Exact Test, where applicable, with significance being defined as p<0.05.

Since all patients entering treatment at our Center sign an initial consent, which permits the use of clinical data for research purposes, as long as the confidentiality of individual patients is maintained, no approval by the Institutional Review Board was required for this study.

Results

A total of 27 IVF cycles with PGD were identified in DOR patients. Amongst those, 19 had undergone IVF without DHEA and 8 with DHEA supplementation. Table 3 summarizes cycle outcomes. As can be seen, peak estradiol levels, oocyte and embryo numbers and the results of PGD, all demonstrated trends towards a beneficial effect of DHEA which did not reach statistical significance, however. Peak estradiol levels were higher and oocyte, as well as embryo numbers, were larger. There was also a trend towards more euploidy in embryos from treated cycles, both in absolute numbers and in percentages of embryos evaluated by PGD.

TABLE 3
IVF cycle and PGD outcomes

| | DHEA-TREATED | DHEA-UNTREATED |
|---|---|---|
| Peak Estradiol ± SD | 2310.3 ± 1108.1 | 2123.3 ± 1054.7 |
| (pg/ml) | | |
| Oocytes ± SD | 10.4 ± 7.3 | 8.5 ± 4.6 |
| Embryos ± SD¹ | 9.1 ± 7.3 | 5.7 ± 2.7 |
| n Euploid ± SD | 2.1 ± 1.4 | 1.6 ± 2.3 |
| % Euploid ± SD | 44.1 ± 37.8 | 21.4 ± 27.5 |
| n Aneuploid ± SD | 4.4 ± 3.0 | 3.5 ± 0.3 |
| % Aneuploid ± SD | 55.9 ± 37.8 | 78.6 ± 27.5 |
| Patients with euploid | 8/8 (100)² | 7/13 (53.8)² |
| embryos (%) | | |

SD, standard deviation of mean;
¹Reflects total number of embryos. Since only high quality 6-cell to 8-cell day-3 embryos undergo PGD, the number of embryos tested for ploidy was smaller.
²Reflects a statistically significant difference by Likelihood ratio (p = 0.004) and (two-sided) Fisher's Exact Test; p = 0.026. Other comparisons in this table did not reach statistical significance.

The only result reaching statistical significance, however, was the difference in the percentage of IVF cycles which resulted in the transfer of at least one euploid embryo, with DHEA treated patients reaching embryo transfer in 100 percent of cycles, while untreated patients did so in only 52.6 percent of cases.

Amongst the 27 reported cycle, three patients contributed pre- and post-DHEA cycles. When these cycles were separately analyzed, they demonstrated similar trends as had been observed for the whole study (Table 4).

TABLE 4
IVF cycle parameters in 3 women with DHEA and -no-DHEA cycles¹

| | |
|---|---|
| Age ± SD (years) | 38.2 ± 5.5 |
| Baseline FSH² ± SD (mIU/ml) | 10.5 ± 1.5 |
| Baseline Estradiol² ± SD (pg/ml) | 54.4 ± 21.7 |

| | DHEA-TREATED | DHEA-UNTREATED |
|---|---|---|
| Time pre-/post DHEA | 2.4 ± 2.5 | 1.9 ± 2.2 |
| (months) | | |
| Oocytes ± SD | 6.0 ± 4.8 | 4.8 ± 1.0 |
| Total Embryos ± SD | 4.0 ± 2.7 | 4.5 ± 0.6 |
| Aneuploid Embryos | 2.0 ± 1.8 | 3.5 ± 0.6 |

SD, standard deviation;
¹None of the differences between the two study groups reached statistical significance,
²Reflects highest baseline level of patients, but not necessarily baseline level during IVF cycle.

Discussion

The here presented study for the first time demonstrates evidence that DHEA improves, to a statistically significant degree, the number of euploid embryos available for embryo transfer after IVF. By doing so, these data also provide for an, at least partial, explanation why DHEA supplementation improves pregnancy chances in women with DOR.

This finding should not surprise since DHEA has been shown not only to improve pregnancy rates, and time to pregnancy, but also to improve egg and embryo quality in such patients. The study, in addition, also demonstrates a trend towards higher percentages of euploid embryos after DHEA and higher absolute numbers of euploid embryos. The relatively small number of DHEA treated patients does not allow, however, at this point to conclude whether DHEA, in an absolute sense, affects embryo ploidy, or not.

The here observed effect of statistically more transferable, euploid embryos, may be due to larger oocyte and embryo numbers, lower aneuploidy rates, or both effects combined. This study does not allow us to differentiate between these possibilities in a statistically valid way. Trends in favor of higher oocyte numbers and lower aneuploidy rates point towards a possible combined effect of DHEA. Prior, larger studies established quite clearly that DHEA increases oocyte yield. A final answer as to the direct effect of DHEA on ploidy will, however, have to await studies of larger patient populations.

The mean number of euploid embyros increased after DHEA treatment by approximately one half embryo (Table 2). This may not appear like very much; however, one half additional embryo, especially if proven euploid, represents significant additional pregnancy potential in women with DOR, who usually produce only relative small embryo numbers. Indeed, in this study this reflects an approximately one third improvement in euploid embryo yield, and results in the availability of at least one embryo for transfer in all post-DHEA cycles, while only 52.6% of untreated cycles achieved the same goal, —a statistically significant difference in embryo transfers. Since aneuploidy (of often morphologically normal appearing embryos) is widely considered a principal cause of IVF failure (and increasing female infertility with advancing age), the here reported finding should not surprise.

As a historical case control study, this study is subjected to potential patient selection biases. Any such biases would, however, affect the study outcomes in favor of negative results: Table 1 demonstrates quite clearly, that, while patient characteristics between DHEA—treated cycles and control cycles did not differ statistically, the trends clearly point towards more severe DOR in women who received DHEA supplementation. This is reflected in older age and higher baseline FSH levels in DHEA patients. Assuming any patient biases, DHEA patients, therefore, should be expected to have fewer euploid embryos and a higher, and not, as here suggested, lower, aneuploidy rate.

How DHEA, and possibly other androgens, generate such improvements on a physiologic level remains to be determined. Based on the incremental improvement in DHEA effects for up to four months, and the correlation of this time span to a full cycle of follicular recruitment, we suspect that DHEA may affect apoptotic processes during follicular recruitment. As a consequence, more healthy follicles survive maturation, reach the stage of gonadotropin sensitivity and become subject to exogenous gonadotropin stimulation. These, in turn, also could be expected to have a higher probability of euploidy.

Other concepts of how androgens may beneficially affect egg and embryo quality have, however, also been proposed. Indeed, androgens, may have in general a direct stimulator effect on the follicle. While earlier studies suggested that higher androgen levels within follicles reflect poor follicle quality, due to a reduced ability of granulosa cells to synthesize estradiol, more recent studies point towards beneficial effects of androgens on oocyte quality. Indeed, androgen receptor expression appears most abundant in healthy pre-antral and antral follicles. Moreover, androgen receptor expression appears to correlate positively with the health of granulosa cells and follicular growth in general, and negatively with granulosa cell apoptosis, potentially also providing support for an effect of DHEA on apoptotic processes within follicular maturation.

The beneficial effects of DHEA and other androgens may vary according to dosage, as the described dose was established by a case study, length of treatment, and androgen utilized in the treatment. The increasing aneuploidy rates with female age are considered the principle cause of decreasing spontaneous female fertility, increasing infertility and rising miscarriage rates. DHEA, or other androgens may improve euploidy rates. Therefore, a mild androgenization of planned conception periods may improve spontaneous female fertility, decrease the rate of female infertility and reduce miscarriage rates in older women.

### EXAMPLE 5

### DHEA Substitution Improves Ovarian Function

A case of probable 17, 20-desmolase deficiency, resulting in abnormally low estradiol, DHEA, androstenedione and testosterone levels, is presented in a woman with a clinical history of, initially, unexplained infertility and, later, prematurely aging ovaries.

This patient started attempting conception in 1996, at age 33. After failing to conceive for over one year, she was diagnosed with hypothyroidism and was placed on levoxyl. She, thereafter, remained euthyroid for the whole period described in this case report. She entered fertility treatment at a prominent medical school based program in Chicago, in August of 1997, where, now age 34, she failed three clomiphene citrate cycles. No further treatment took place until a laparoscopy was performed in October of 1999, at a prominent Atlanta-based infertility center (where the couple had relocated to), revealing stage II endometriosis which was laser vaporized. Following surgery, a fourth clomiphene citrate cycle and a first gonadotropin-stimulated cycle failed. Table 5 presents selected key lab data for all ovarian stimulation cycles the patient underwent. A first in vitro fertilization (IVF) cycle was performed, at age 36, in October of 2000.

This cycle resulted in expected oocyte and embryos yields. Three embryos were transferred, resulting in a chemical pregnancy. Three other embryos were cryopreserved. However, because of a persistently thin endometrium, a number of attempts at transfer were cancelled.

In April of 2001, the patient was, based on an abnormal glucose tolerance test, diagnosed with insulin resistance, and was placed on metformin, 500 mg thrice daily. She had no signs of polycystic ovarian disease: her ovaries did not look polycystic, she was not overweight, had no signs of hirsutism or acne, and androgen, as well as estradiol, levels were in a low normal range (Table 2). In June of 2001 (age 37), a second IVF cycle was initiated. In this cycle the patient demonstrated the first evidence of ovarian resistance to stimulation in that she produced only six oocytes. Only one out of five mature oocyte fertilized, despite the utilization of intracytoplasmic sperm injection (ICSI). The previously cryopreserved embryos were, therefore, thawed and transferred, together with the one fresh embryo from the current cycle. The transfer was unsuccessful.

In August of 2001, the female's FSH level for the first time was abnormally elevated (11.4 mIU/ml), with estradiol levels remaining low-normal. Subsequent FSH levels were 19.1, 9.7 and 9.8 mIU/ml in November and December (twice), respectively, all with low-normal estradiol levels. FSH levels continued to fluctuate in 2002, with levels reported as 11.4 mIUI/ml in February, 8.7 in March, 13.6 in June and 19.6 in September, while estradiol levels remained persistently low-normal (Table 2).

A third IVF cycle was started in October of 2002, with a baseline FSH of 11.3 mIUI. Ovarian stimulation, which in the prior two cycles had been given with only recombinant FSH (and antagonists), was now given in a combination of recombinant FSH and hMG at a combined dosage of 300 IU daily. Estradiol levels reached only 890 pg/ml and only 5 oocytes were retrieved. All four mature oocytes fertilized and four embryos were transferred. A twin pregnancy was established by ultrasound and a singleton by heart beat. This pregnancy was, however, miscarried and confirmed as aneuploid with a Trisomy 22.

The fact that this cycle, after the addition of hMG to the stimulation protocol, appeared more successful, led the patient to a search of the medical literature. Like our previously reported patient (Barad and Gleicher, 2005), this patient discovered the case series reported by Casson and associates (Casson, et al., 2000). The paper attracted the patient's interest. In follow up, she asked a medical endocrinologist to evaluate her adrenal function. An initial evaluation revealed very low DHEA, DHEA-S, androstenedione and testosterone levels (Table 2). An ACTH-stimulation test was ordered which showed the expected increase in cortisol level, but unchanged, low DHEA. DHEA-S and testosterone levels (Table 3). The patient was advised by her medical endocrinologist that the most likely explanation for such a finding was a 3-beta hydroxysteroid dehydrogenase deficiency. This enzyme defect is, however, associated with an accumulation of DHEA and, therefore, high levels of the hormone. (Speroff et al., 1999a). Such a diagnosis for the patients is, therefore, unlikely. Instead, as FIG. 1 demonstrates, abnormal 17,20-desmnolase (P450c 17) function would be expected to result in exactly the kind of hormone profile, reported in this patient after ACTH stimulation, characterized by persistently low DHEA, androstenedione, testosterone and estradiol levels, but normal aldosterone and cortisol levels.

In July of 2003, the patient was started on 25 mg daily of micronized DHEA. After five weeks of treatment, DHEA DHEA-S and androstenedione levels had normalized into mid-ranges. (Even though androstenedione is partially produced through the activity of 17,20-desmolase from 17-hydroxyprogesterone, part is also derived from DHEA through the activity of 3-beta hydroxysteroid dehydrogenase [Speroff et al., 1999a]. The normalization of andostenedione, after DHEA administration, therefore, also speaks for an underlying 17,20-desmolase defect, and not a 3-beta hydroxysteroid dehydrogenase deficiency.) In the third and fourth month, following the start of DHEA supplementation, the patient ovulated spontaneously with estradiol levels of 268 and 223 pg/ml (Table 2), respectively. measured on the day of LH surge.

On Jan. 28, 2004 (age 39), and after DHEA therapy of approximately six months, a fourth IVF cycle was initiated. Her baseline FSH level in that cycle was 9.6 mIU/ml, estradiol 56 pg/ml. Stimulation took place with 300 IU of recombinant FSH (without hMG) and with an agonist flare protocol. Estradiol levels reached a peak of 1764 pg/ml, 8 oocytes were retrieved, six out of seven mature oocytes fertilized and six embryos were transferred. A triplet pregnancy was established with heart beats. Two, out of the three fetuses lost heart beat spontaneously, and the patient delivered by cesarean section, at term, a healthy singleton male infant.

At surgery, her ovaries were closely inspected and described as “old” and “small”, with the left one being described as “almost dead.” DHEA and DHEA-S levels at six months of pregnancy were reported at “record lows.” DHEA-S, six weeks post-delivery, was still very low (Table 5). At time of this report, the male offspring is nine months old and the mother has been re-started on DHEA in an attempt at another pregnancy.

DHEA substitution resulted in apparently normal peripheral DHEA levels, spontaneous ovulation and normal estradiol production by the ovaries. An IVF cycle, after approximately six months of DHEA substitution, showed, in comparison to a pre-DHEA IVF cycle, improved peak estradiol levels, increased oocyte and embryo numbers and resulted, at age 39, after 6 years of infertility therapy, in a triplet pregnancy and a normal singleton delivery.

Low DHEA levels appear associated with female infertility and ovarian aging. DHEA substitution normalizes peripheral DHEA levels and appears to improve ovarian response parameters to stimulation.

The reported patient exhibited some of the classical signs of prematurely aging ovaries (Nikolaou and Templeton, 2003; Gleicher N, 2004) which include ovarian resistance to stimulation, poor egg and embryo quality and prematurely elevated FSH levels.

We have previously suggested that the decrease in DHEA levels, with advancing female age, may be an inherent part of the ovarian aging process and may, at least in part, and on a temporary basis, be reversed by external DHEA substitution (Barad and Gleicher, 2005, 2005a). This case demonstrates that low DHEA levels are, indeed, associated with all the classical signs of both prematurely and normally aging ovaries. While association does not necessarily suggest causation, the observed sequence of events in this patient supports the notion that low DHEA levels adversely affect ovarian function.

The patient was initially thought to have largely unexplained infertility. Approximately 10 percent of the female population is believed to suffer from premature aging ovaries and this diagnosis is, indeed, often mistaken for unexplained infertility (Nikolaou and Templeton, 2003, Gleicher N, 2005). She later developed quite obvious signs of prematurely aging ovaries and, finally, even showed elevated FSH levels. In the time sequence, in which all of these observations were made, the patient followed the classical parallel, premature aging curve we, and others, have previously described (Nikolaou and Templeton, 2003; Gleicher N, 2005).

Once substituted with oral DHEA, a reversal of many findings characteristic of the aging ovary, was noted. First, the patient's DHEA and DHEA-S levels normalized. In subsequent natural cycles an apparently normal spontaneous follicular response was observed, with normal ovulatory estradiol levels in a patient with persistently low estradiol levels before DHEA treatment (Table 5). The response to ovarian stimulation improved, quantitatively and qualitatively, as the patient improved peak estradiol levels, oocyte and embryo numbers and, as the successful pregnancy may suggest, also embryo quality.

A case report can, quite obviously, not be seen as confirmation for all of these observations. Moreover, one cannot preclude that other factors contributed. For example, the ovarian stimulation protocol had switched from an antagonist to an agonist flare protocol. Yet, our previously reported data quite convincingly demonstrates that DHEA supplementation in women with aging ovaries, indeed, to a statistical degree, improves oocyte yield and egg as well as embryo quality (Barad and Gleicher, 2005a). Our data also suggest that DHEA may improve pregnancy rates and reduce aneuploidy rates in embryos from older women, though the latter two outcome modifications are not yet statistically robust. Finally, our data have demonstrated that a maximal effect of DHEA is achieved after at least about four consecutive months of use (Barad and Gleicher, 2005a). This patient was on DHEA treatment for approximately six months before she conceived the pregnancy that led to her first live birth.

This case is unusually well documented in its DHEA deficiency and in its most likely cause. We are considering the diagnosis of 17,20-desmolase deficiency as very likely, though not absolutely proven, since only a tissue diagnosis, or adrenal outflow cannulation, can offer absolute proof of an adrenal enzyme defect. Neither procedure has, so far, been performed in this case. The reported adrenal response to ACTH stimulation (Table 5) allows, however, no other explanation (FIG. 1).

TABLE 5
Relevant laboratory results

| Date | TEST | RESULT (Normal values)* | COMMENTS |
|---|---|---|---|
| August 1997 | TSH | 7.8 mlU/l (0.4-5.5) | Diagnosis of |
| | | | hypothyroidism |
| May 1999 | FSH | 4.0 mIU/ml | |
| April 2001 | Glucose tolerance test | Elevated 1/2 hour insulin levels | Diagnosis of |
| | | Normal Glucose levels | insulin resistance |
| June 2001 | FSH | 7.7 mIU/ml | |
| | Estradiol | 33 pg/ml | |
| August 2001 | Testosterone free/weakly bound | 2 ng/dl (3-29) | Diagnosis of |
| | free only | 1 pg/ml (1-21) | prem. ov. aging |
| | total | 13 ng/dl (15-70) | |
| | DHEA-S | 96 mcg/dl (12-379) | |
| | Total Cortisol | 14.2 mcg/ml (4-22) | |
| | FSH | 11.4 mIU/ml | |
| | Estradiol | 45 pg/ml | |
| October 2001 | Estradiol periovulatory | 119 pg/ml | |
| November 2001 | Testosterone total | 23 ng/ml (14-76) | |
| | Androstenedione | 98 ng/ml (65-270) | |
| | Ovarian antibodies | negative | |
| | FSH | 19.1 mIU/ml | |
| | Estradiol | 23 pg/ml | |
| December 2001 | FSH | 9.7 mIU/ml | |
| | Estradiol | 27 pg/ml | |
| February 2002 | Testosterone total | <20 ng/dl (20-76) | |
| | Androstenedione | 76 ng/dl (65-270) | |
| | FSH | 11.4 mIU/ml | |
| | Estradiol | 28 pg · ml | |
| March 2002 | Testosterone total | 16 ng/dl (15-70) | |
| | FSH | 8.7 mIU/ml | |
| | Estradiol | 29 pg/ml | |
| May 2002 | FSH | 13.6 mIU/ml | |
| | Estradiol | 30 pg/ml | |
| | periovulatory | 139 pg/ml | |
| June 2002 | periovulatory | 50 pg/ml | |
| September 2002 | Testosterone total | 15 ng · dl (15-70) | |
| | free | 1.6 pg/ml (1-8.5) | |
| | % free | 0.0107 (0.5-1.8) | |
| | Estradiol periovulatory | 136 pg/ml | |
| October 2002 | FSH | 11.3 mIUI/ml | |
| | Estradiol | 43 pg/ml | |
| February 2003 | FSH | 13.6 mIU/ml | |
| | Estradiol | 33 pg/ml | |
| March 2003 | FSH | 8.9 mIU/ml | |
| | Estradiol | 67 pg/ml | |
| May 2003 | Anti-adrenal antibodies | negative | |
| | Estradiol periovulatory | 139 pg/ml | |
| | DHEA | 132 ng/dl (130-980) | |
| | DHEA-S | 79 mcg/dl (52-400) | |
| | Testosterone total | 34 ng/dl (20-76) | |
| | free | 3 pg/ml (1-21) | |
| July 2003 | DHEA TREATMENT START | | |
| | DHEA | 296 ng/dl (130-980) | |
| | DHEA-S | 366 mcg/dl (52-400) | |
| | Androstenedione | 121 ng/dl (65-270) | |
| September 2003 | Estradiol periovulatory | 268 pg/ml | |
| October 2003 | FSH | 14.7 mIUI/ml | |
| | Estradiol | 44 pg/ml | |
| | periovulatory | 224 pg/ml | |
| November 2003 | FSH | 17 mIU/ml | |
| | Estradiol | 38 pg/ml | |
| December 2003 | DHEA | 278 ng/ml (130-980) | |
| | DHEA-S | 270 mcg/dl (52-400) | |
| | Testosterone total | 25 ng/ml (20-76) | |
| | free and weekly bound | 4 ng/dl (3-29) | |
| | free | 2 pg/ml (1-21) | |
| January 2004 | FSH | 18 mIU/ml | 4^{th} IVF |
| | FSH | 9.6 mIU/ml | CYCLE START |
| | Estradiol | 56 pg/ml | |
| August 2004 | MID_PREGNANCY DHEA | 74 ng/dl (135-810) | |
| | DHEA-S | 27 mcg/dl (**) | |
| October 2004 | | | DELIVERY |
| December 2004 | DHEA-S | 52 mcg/dl (44-352) | |

*Laboratory tests were performed at varying laboratories
** No pregnancy levels available from laboratory

TABLE 6
ACTH stimulation test

| HORMONE | BASELINE | +30 MINUTES | +60 MINUTES |
|---|---|---|---|
| DHEA-S (mcg/ml) | 87 | 88 | 83 |
| Cortisol total (mcg/dl) | 15 | 26 | 27 |
| Testosterone total (ng/dl) | 28 | 32 | 33 |
| free and weakly bound | 5 | 5 | 5 |
| free | 3 | 3 | 3 |

This case is also remarkable in that it includes evidence of ovarian, thyroid, pancreatic and adrenal dysfunction in one patient. Such a combination of glandular involvements has been reported in the autoimmune polyglandular syndrome(s), characterized by combined end-organ involvements in an autoimmune assault of thyroid, parathyroid, adrenal, pancreas, ovary and, at times, other organs. It appears that at least some of these cases are inherited in Mendelian fashion as an autosomal recessive disorder (Consortium, 1997). End-organ function may be vulnerable to autoantibody attacks with various cross-reactivities. For example, women diagnosed with both adrenal and ovarian insufficiency have been shown to demonstrate antibody activity against P450scc, the adrenal enzyme essential to steroidogenesis (Winqvist et al., 1995). It has been suggested that any one of the vital enzymes, involved in steroidogenesis, may be vulnerable to autoimmune inactivation. (Speroff et al., 1999b).

Considering this patient's hypothyroidism, insulin resistance, premature ovarian aging process and, quite obviously, selective adrenal insufficiency, she deserves close observation in regards to the possible appearance of other characteristic features of the autoimmune polyglandular syndrome(s). It is also noteworthy that she reports a family history in support of a potential genetic predisposition: Her brother's son has been diagnosed with congenital adrenal hyperplasia, which can be caused by 21-hydroxilase (P450c21)-, 11 beta hydroxylase (P450c11-beta)- or 3-beta hydroxysterod dehydrogenase deficiencies (Speroff et al., 1999a). And her father required testosterone substitution to initiate puberty.

This case report also presents further evidence for DHEA deficiency as a cause of female infertility and as a possible causative agent in the aging processes of the ovary. It also presents further confirmation of the value of DHEA substitution whenever the suspicion exists that ovaries may be lacking of DHEA substrate. Finally, this case report raises the important question what the incidence of adrenal 17,20-desmolase (P450c17) deficiency is in women with prematurely aging ovaries. Why ovaries age prematurely is, in principle, unknown. Since the process is familial (Nikolaou and Templeton, 2003), it is reasonable to assume that, like other adrenal enzymatic defects, 17,20-desmolase deficiency, may occur in either a sporadic or an inherited form. As both forms will result in abnormally low DHEA levels, both may then, indeed, lead to phenotypical expression as premature ovarian aging.

### EXAMPLE 6

### Increase Male Fetus Sex Ratio

Androgenization of females with dehydroepiandrosterone (DHEA), as we recently have been utilizing in the fertility treatment of women with diminished ovarian reserve, in combination with the investigation of spontaneous, versus in vitro fertilization (IVF)—conceived, pregnancies allows for an investigation of the basic theory of sex allocation and its possible pathophysiologic mechanisms.

The treatment protocol for long-term supplementation with DHEA that may improve oocyte and embryo quantity, quality, pregnancy rates and time to conception in women with diminished ovarian reserve involves 25 mg of micronized, pharmaceutical grade DHEA, TID will usually uniformly raise levels of unconjugated DHEA above about 350 ng/dl, and, therefore, raise baseline testosterone. Estradiol baseline levels may also be raised.

A retroactive review of either ongoing or delivered pregnancies beyond 20 weeks gestational age, conceived while on DHEA treatment for at least 60 days, revealed 23 women. They were contacted by a staff person and queried whether a delivery had already taken place, or not. If a delivery had taken place, the gender of each delivered child was recorded. If the pregnancy was still undelivered, the patient was queried whether she had undergone an amniocentesis or chronic villous biopsy and the results for the genetic test were recorded for each fetus. A total of 19 women were reached and reports on 16 singleton and 3 twin pregnancies were recorded.

In addition, the medical records of all 19 women were reviewed in order to determine whether they conceived spontaneously, defined as including pregnancies conceived with intrauterine inseminations, or by IVF. If conception had occurred by IVF, we recorded whether fertilization was spontaneous or by intracytoplasmic sperm injection (ICSI).

As additional control group, we selected seven women, who had undergone one IVF cycle with preimplantation genetic diagnosis (PGD), while for at least 60 days on DHEA supplementation, but had not conceived. The PGD data, defining each embryo's gender, were recorded. Statistics were performed using a binomial runs test, comparing seen distributions with an expected distribution of 50 percent, with p<0.05 defining significance.

As a result, sixteen singleton pregnancies resulted in 11 males and 5 females (N.S.). Two of three twin pregnancies were heterozygous and one homnozygous. If outcomes of both heterozygous twins, but of only one homozygous twin, were added, the final gender distribution was 15 males and 6 females (p=0.078, N.S.)

Amongst six pregnancies, spontaneously conceived, the distribution between female and male offspring was equal, at three and three, respectively. Whereas amongst the remaining 15 offspring, which were products of pregnancies achieved through IVF, the distribution was 12 males and 3 females (p=0.035). Only one IVF patient failed to have ICSI. Amongst women undergoing IVF and PGD, 53 embryos were analyzed from 17 IVF cycles, all having undergone ICSI. The gender distribution was not significantly skewed, with 27 being male and 26 female.

This study allows for the dissection of the conception process into its various stages and, therefore, permits an analysis of, not only the basic question whether androgenization does indeed, affect gender selection in the human, but also how such a selection may be influenced.

The here presented data, demonstrating a strong trend towards significance overall, and significance (p=0.035) amongst IVF patients, suggest, convincingly that gender determination may be influenced by hormonal environment. Women with evidence of androgen excess, due to either polycystic ovarian syndrome (PCOS) or incomplete adrenal hyperplasia, would appear ideally suited study subjects for such follow up studies. Assuming an effect of androgens on gender selection, such women should give birth to a preponderance of male offspring. Confirming such a finding could present a potential additional explanation for the evolutionary preservation of PCOS in practically all human races.

Even though the number of spontaneously conceived pregnancies was very small, and, therefore, a type-2 error cannot be ruled out, the even distribution of gender in this group of patients argues against a selection process towards male, which is driven by the follicular environment, as was suggested by Grant and Irwin (2005). The even distribution of gender in preimplantation embryos, seen in the control group, speaks against such an effect,

While the foregoing written description of the invention enables one of ordinary skill to make and use what is considered presently to be the best mode thereof, those of ordinary skill will understand and appreciate the existence of variations, combinations, and equivalents of the specific exemplary embodiments thereof. The invention is therefore to be limited not by the exemplary embodiments herein, but by all embodiments within the scope and spirit of the appended claims.

What is claimed is:

1. A method of increasing the number of euploid oocytes per cycle, comprising:
administering an androgen to a female for at least about one month; and
harvesting oocytes from said female.

2. A method according to claim 1, wherein said androgen comprises dehydroepiandrosterone.

3. A method according to claim 2, wherein said female is human.

4. A method according to claim 3, wherein between about 50 mg and about 100 mg per day of said dehydroepiandrosterone is administered to said female.

5. A method according to claim 3, wherein between about 15 mg and about 40 mg of said dehydroepiandrosterone is administered three times a day to said female.

6. A method of increasing male fetus sex ratio comprising raising baseline androgen levels in a female.

7. A method according to claim 6, wherein said raising step occurs prior to blastocyst implantation.

8. A method according to claim 6, wherein said raising step occurs at about the time of blastocyst implantation.

9. A method according to claim 6, wherein said raising step occurs after blastocyst implantation.

10. A method according to claim 7, wherein said blastocyst is the product of an in vitro fertilization process.

11. A method according to claim 6, further comprising raising baseline estrogen levels in said female.

12. A method according to claim 6, wherein said androgen is testosterone.

13. A method according to claim 6, wherein said androgen is dehydroepiandrosterone.

14. A method according to claim 13, wherein said baseline dehydroepiandrosterorne level is above about 250 ng/dl.

15. A method according to claim 13, wherein said baseline dehydroepiandrosterone level is above about 350 ng/dl.

16. A method according to claim 6, wherein said raising baseline androgen levels step is accomplished by administering dehydroepiandrosterone.

17. A method according to claim 16, wherein said dehydroepiandrosterone administration comprises between about 50 and about 100 mg per day of said dehydroepiandrosterone.

18. A method according to claim 16, wherein said dehydroepiandrosterone administration comprises between about 15 mg and about 40 mg of said dehydroepiandrosterone administered about three times a day.

19. A method according to claim 16, wherein said administering dehydroepiandrosterone is for at least about one month.

20. A method of reducing miscarriages in females comprising administering an androgen for at least about one month.
